(19) United States
(12) Patent Application Publication
(10) Pub. No.: US 2017/0315222 A1
(43) Pub. Date: Nov. 2, 2017

(54) METHOD OF MANUFACTURING ELECTRONIC DEVICE AND ELECTRONIC DEVICE

(71) Applicant: FUJIFILM Corporation, Tokoy (JP)

(72) Inventors: Seigo NAKAMURA, Kanagawa (JP); Yoshihisa USAMI, Kanagawa (JP)

(73) Assignee: FUJIFILM Corporation, Tokyo (JP)

(21) Appl. No.: 15/648,682

(22) Filed: Jul. 13, 2017

Related U.S. Application Data
(63) Continuation of application No. PCT/JP2016/051380, filed on Jan. 19, 2016.

(30) Foreign Application Priority Data
Jan. 21, 2015 (JP) 2015-009110

(51) Int. Cl. G01S 13/42; H04L 5/00; G01R 29/08; H04L 27/26; A61B 5/107

(52) U.S. Cl. CPC G01S 13/42; H04L 27/2655; H04L 5/0073; A61B 5/107; H04L 5/0035; G01R 29/08; H04L 5/0048

(57) ABSTRACT

After an electroconductive projection is formed on an electrode of an electronic element, a gas barrier film on which an adhesive layer and a contact hole are formed is laminated and pressure-bonded onto a substrate on which the electronic element is formed. Alternatively, after a gas barrier film on which an adhesive layer and a contact hole are formed is laminated on a substrate on which an electronic element is formed and an electroconductive projection is formed on the electrode inside the contact hole, the substrate and the gas barrier film are pressure-bonded to each other, and the contact hole is filled with an electroconductive material. In this manner, there are provided a method of manufacturing an electronic device; and an electronic device to which a take-out wire used to reliably connect the electronic device to an external device using a small contact hole can be connected even in a case where the electronic device is small.

### CROSS-REFERENCE TO RELATED APPLICATIONS

This application is a Continuation of PCT International Application No. PCT/JP2016/051380 filed on Jan. 19, 2016, which claims priority under 35 U.S.C. §119(a) to Japanese Patent Application No. 2015-009110 filed on Jan. 21, 2015. The above application is hereby expressly incorporated by reference, in its entirety, into the present application.

### BACKGROUND OF THE INVENTION

### 1. Field of the Invention

The present invention relates to a method of manufacturing an electronic device and an electronic device such as an organic EL device or an organic TFT. Specifically, the present invention relates to a method of manufacturing an electronic device and an electronic device obtained by sealing an electronic element with a gas barrier film.

### 2. Description of the Related Art

As various electronic elements, organic electronic elements such as organic EL elements (organic electroluminescence elements) and organic thin film transistors (organic TFTs) have been developed.

Electronic elements are typically vulnerable to moisture or oxygen. Among the electronic elements, organic electronic elements are severely degraded due to moisture.

Therefore, electronic elements are formed and then sealed with a sealing layer which prevents permeation of moisture or gas. Here, from the viewpoint of productivity, a gas barrier film is considered to be used as a sealing layer. In other words, a plurality of electronic elements can be sealed at once by forming the plurality of electronic elements on a substrate and bonding a gas barrier film to the substrate using an adhesive.

In a case where an electronic element is sealed with a gas barrier film, it is necessary to take out a wire for connecting the electronic element to an external device.

In a case of a large-sized display, a wire is typically taken out from the peripheral portion thereof.

Meanwhile, in a case of a small-sized electronic element, a wire is unlikely to be taken out from the peripheral portion thereof. In this case, it is considered that a contact hole for a wire, which penetrates through a gas barrier film and an adhesive layer and is used to take out a wire, is formed and a take-out wire for connecting the electronic element to an external device is provided in the contact hole.

For example, JP2011-62958A discloses a method of continuously supplying a film composite which includes a gas barrier film and an adhesive layer; performing punching processing or slitting processing on a part of the film composite to form a contact hole (portion for taking out a wire); and continuously laminating the film composite, in which the contact hole is formed, on a substrate on which electronic elements are formed through roll lamination, in which the continuous supply of the film composite, the formation of the contact hole, and the attachment to the roll are performed in-line.

### SUMMARY OF THE INVENTION

According to the method described in JP2011-62958A, the electronic element can be sealed using a gas barrier film provided with a contact hole for a take-out wire with high production efficiency by means of using a so-called roll to roll system.

There are various electronic elements with various sizes, for example, large-sized electronic elements such as a display and small-sized electronic elements such as an IC tag.

In a case where an electronic element is small, it is necessary for a contact hole to be small according to the size of the electronic element. Particularly in a case of a small-sized electronic element such as an IC tag, there has been a demand for miniaturization in recent years. In accordance with this demand, a contact hole is required to be small.

Further, when an effective area of an electronic element, such as a display area of a display, is considered, it is preferable that the contact hole is small even if the electronic element is large.

As described in JP2011-62958A, the gas barrier film on which the adhesive layer is formed is bonded to the substrate on which the electronic element is formed by typically performing pressure-bonding, in which the gas barrier film and the substrate are laminated on each other and pressed against each other. In addition, the adhesive layer is heated or irradiated with light as necessary when the pressure-bonding is performed.

Here, when the gas barrier film and the substrate are pressure-bonded to each other, an adhesive moves so as to fill the contact hole. However, the contact hole is blocked by the adhesive in a case where the contact hole is small, and thus a wire cannot be taken out.

The present invention has been made to solve the above-described problems of the related art and an object thereof is to provide a method of manufacturing an electronic device and an electronic device in which a wire can be stably taken out from a contact hole for forming a take-out wire that connects the electronic device to an external device even in a case where the contact hole is small in the electronic device formed by sealing an electronic element with a gas barrier film.

In order to achieve the above-described object, according to a first aspect of the present invention, there is provided a method of manufacturing an electronic device comprising: a process of forming an adhesive layer on a gas barrier film and forming a contact hole penetrating through the gas barrier film and the adhesive layer; a process of forming a projection having electroconductivity on an electrode of an electronic element on a substrate provided with at least one electronic element; and a process of aligning the contact hole with the projection, allowing the adhesive layer and the surface on which the electronic element is formed to face each other, laminating the substrate and the gas barrier film on each other such that the substrate and the gas barrier film are pressure-bonded to each other, in which the following Expressions (1) and (2) are satisfied when the size of the contact hole is set as X [μm], the height of the projection is set as Y [μm], and the thickness of the adhesive is set as L [μm].

0<\sqrt{X}<\sqrt{5000} Expression (1)

Y>−0.018×L×(\sqrt{X}−\sqrt{5000}) Expression (2)

Further, according to a second aspect of the present invention, there is provided a method of manufacturing an electronic device comprising: a process of forming an adhesive layer on a gas barrier film and forming a contact hole penetrating through the gas barrier film and the adhesive layer; a process of aligning the contact hole with an electrode of an electronic element on a substrate provided with at least one electronic device, allowing the adhesive layer and the surface on which the electronic element is formed to face each other, and laminating the substrate and the gas barrier film on each other; a process of forming a projection having electroconductivity on the electrode of the electronic element inside the contact hole; and a process of pressure-bonding the substrate and the gas barrier film to each other, in which the following Expressions (1) and (2) are satisfied when the size of the contact hole is set as X [μm], the height of the projection is set as Y [μm], and the thickness of the adhesive is set as L [μm].

0<\sqrt{X}<\sqrt{5000} Expression (1)

Y>−0.018×L×(\sqrt{X}−\sqrt{5000}) Expression (2)

In the method of manufacturing an electronic device of the present invention, it is preferable that the size of a largest portion of the projection is smaller than the size of the contact hole.

Further, it is preferable that the height of the projection is greater than the thickness of the adhesive layer.

Further, it is preferable that the size of the projection becomes gradually smaller toward the upside in a height direction.

Further, it is preferable that the method of manufacturing an electronic device further comprises a process of filling the contact hole with an electroconductive material.

Further, it is preferable that the gas barrier film and the substrate have flexibility.

Further, it is preferable that at least one of the formation of the adhesive layer, the formation of the contact hole, the formation of the projection, the lamination of the substrate and the gas barrier film on each other, or the pressure-bonding of the substrate and the gas barrier film to each other is performed using a lone substrate and a long gas barrier film while at least one of the substrate or the gas barrier film is conveyed in a longitudinal direction.

There is provided an electronic device comprising: a substrate; at least one electronic element which is formed on the substrate; a gas barrier film which seals the electronic element; an adhesive layer which bonds the gas barrier film to the substrate; a contact hole which penetrates through the gas barrier film and the adhesive layer and is formed in a position corresponding to an electrode of the electronic element; and a take-out wire which passes through the contact hole and is connected to the electrode of the electronic element, in which the contact hole is filled with the take-out wire and the take-out wire includes a size varying portion.

In the above-described electronic device of the present invention, it is preferable that the take-out wire includes a narrowed portion which becomes gradually smaller toward the upside and becomes gradually larger toward the upside from a smallest portion.

Further, it is preferable that a plurality of the electronic elements are formed on the substrate.

According to the present invention, it is possible to stably take out a wire from a contact hole for taking out a wire even in a case where the contact hole is small in an electronic device formed by sealing an electronic element with a gas barrier film.

### BRIEF DESCRIPTION OF THE DRAWINGS

FIG. 1 is a view schematically illustrating an example of an electronic device of the present invention.

FIG. 2 is a view schematically describing a method of manufacturing an electronic device of the present invention.

FIG. 3 is a view schematically describing a method of manufacturing an electronic device of the present invention.

### DESCRIPTION OF THE PREFERRED EMBODIMENTS

Hereinafter, a method of manufacturing an electronic device and an electronic device of the present invention will be described in detail based on preferred embodiments shown in the accompanying drawings.

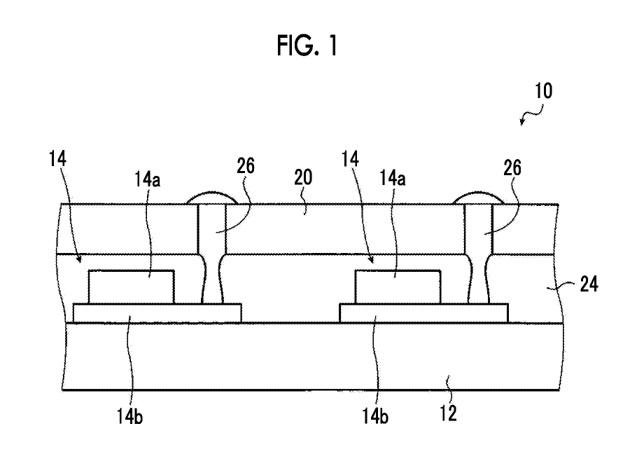

FIG. 1 schematically illustrates an example of an electronic device of the present invention.

An electronic device 10 illustrated in FIG. 1 basically includes a substrate 12, an electronic element 14, a gas barrier film 20, an adhesive layer 24, and a take-out wire 26. In the example shown in the figure, the electronic element 14 is configured of an electronic element main body 14a and an electrode 14b. This electronic device 10 is manufactured according to the method of manufacturing an electronic device of the present invention.

In the example shown in the figure, a plurality of electronic elements 14 are formed on one substrate 12.

The present invention is not particularly limited to this configuration, and only one electronic element 14 may be formed on one substrate 12. However, from the viewpoint of productivity, it is preferable that the substrate 12 includes a plurality of electronic elements 14.

In the present invention, the electronic element 14, that is, the electronic device 10 is not particularly limited, and various known electronic elements 14 can be used. Among these, the electronic element 14 prepared by utilizing an organic semiconductor is preferably used.

Examples thereof include organic EL elements such as an organic EL display and an organic EL lighting; devices such as an RFID tag formed of a logic circuit having an organic TFT; various sensors for which an organic TFT is used; photoelectric conversion elements such as an organic solar cell; and organic thermoelectric conversion elements.

Further, the electrode 14b is a known electrode provided in a known electronic element.

The electronic element 14, that is, the electronic element main body 14a and the electrode 14b may be formed using a known method.

The substrate 12 is a known material used for various electronic elements 14 (electronic device 10) and various substrates such as a sheet-like material (film) or a plate-like material which has insulating properties can be used.

Specific examples thereof include a resin such as polyethylene (PE), polypropylene (PP), polyethylene terephthalate (PET), polyethylene naphthalate (PEN), polyimide (PI), a cycloolefin copolymer (COC), or a cycloolefin copolymer (COP); a metal provided with an insulating film on the surface (aluminum foil or the like); and a sheet-like material or a plate-like material formed of glass or ceramics.

Further, a gas barrier film similar to the gas barrier film 20 described below can be preferably used as the substrate 12.

The thickness of the substrate 12 may be suitably set according to the size or the type of the electronic device 10 to be prepared.

It is preferable that the substrate 12 has flexibility. A typical gas barrier film 20 has flexibility. Therefore, when the substrate 12 has flexibility, the method of manufacturing an electronic device can be performed using a so-called roll to roll (hereinafter, also referred to as RtoR) system.

The gas barrier film 20 is a known gas barrier film obtained by forming a gas barrier layer on a support.

Various known gas barrier films can be used as the gas barrier film 20. However, since a gas barrier film having an electroconductive layer such as aluminum foil is electroconductive with the take-out wire 26, a gas barrier film formed of an inorganic oxide or an inorganic nitride is preferable. An organic-inorganic lamination type gas barrier film obtained by forming, on a support formed of a plastic film or the like, one or more combinations of an inorganic layer formed of silicon nitride or the like and an organic layer which serves as an underlying layer of the inorganic layer and is formed of an acrylic resin or a methacrylic resin may be exemplified. In the organic-inorganic lamination type gas barrier film, the uppermost layer may be an organic layer or an inorganic layer.

As the organic-inorganic lamination type gas barrier film, the configuration described in paragraphs [0011] to [0030] of JP2009-094051A may be exemplified.

The thickness of the gas barrier film 20 may be suitably set according to the size or the type of the electronic device 10 to be prepared.

Further, for the same reason as for the substrate 12, it is preferable that the gas barrier film 20 has flexibility. In addition, a typical gas barrier film has flexibility.

The adhesive layer 24 is formed by bonding the gas barrier film 20 to the substrate 12 on which the electronic element 14 is formed.

As the adhesive layer 24, various adhesives capable of bonding the gas barrier film 20 to the substrate 12 on which the electronic element 14 is formed can be used. Examples of the adhesives include a heat sealing agent, a heat-sensitive adhesive, a pressure-sensitive adhesive, and a photosensitive adhesive. In addition, an epoxy-based adhesive having excellent gas barrier properties is preferable as the material for forming the adhesive layer 24.

The take-out wire 26 is used to connect the electrode 14b of the electronic element 14 to an external device such as a power source or a drive circuit, provided from the electrode 14b to the upper surface of the gas barrier film 20 (surface on a side opposite to the substrate 12) passing through the adhesive layer 24 and gas barrier film 20.

Although described in the description of the method of manufacturing an electronic device later, in the electronic device 10 of the present invention which is manufactured by the method of manufacturing an electronic device of the present invention, the take-out wire 26 includes a size varying portion in the height direction. It is preferable that that the take-out wire 26 includes a narrowed portion which becomes gradually smaller toward the upside and becomes gradually larger toward the upside from a smallest portion.

In the present invention, the upside indicates a direction toward the gas barrier film 20 from the substrate 12. In addition, the size of the take-out wire 26 in the present invention indicates the size in the direction perpendicular to the height direction, that is, the thickness direction of the adhesive layer 24 and the gas barrier film 20, that is, the vertical direction.

In other words, in a case where the take-out wire 26 has a shape of a solid of revolution such as a cylinder or a cone, the take-out wire 26 includes a diameter varying portion in the height direction, that is, the extending direction of a central line and preferably includes a narrowed portion having a diameter which is gradually decreased toward the upside and is gradually increased toward the upside from a minimum diameter portion.

The take-out wire 26 may be formed of known electroconductive materials, for example, metals such as silver, gold, aluminum, copper, platinum, lead, zinc, tin, and chromium and carbon.

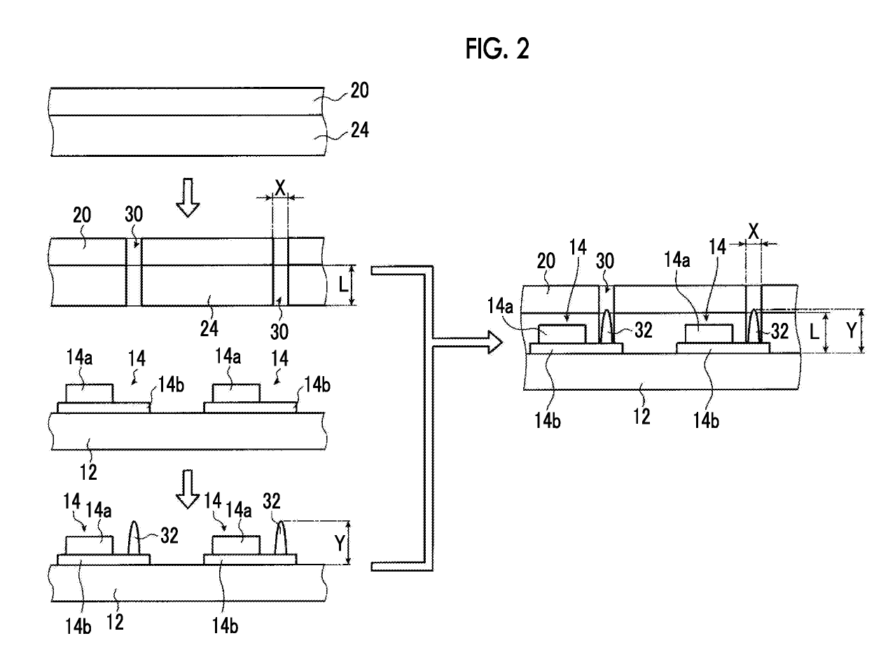

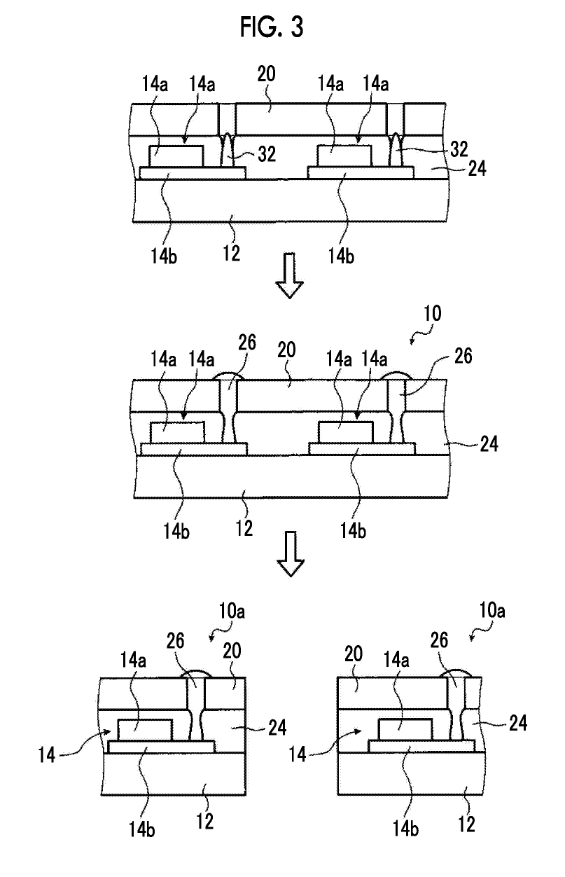

Hereinafter, the present invention will be described in more detail by describing the method of manufacturing an electronic device of the present invention with reference to FIGS. 2 and 3.

First, as shown in the upper left stage of FIG. 2, the adhesive layer 24 is formed on the gas barrier film 20. A thickness L of the adhesive layer 24 will be described later.

The adhesive layer 24 may be formed using a known method according to the material of forming the adhesive layer 24 or the thickness thereof. Examples of the known method include a method of applying an adhesive which forms the adhesive layer 24, drying the applied adhesive, and optionally further, semi-curing the resultant and a method of forming the adhesive layer by performing adhesion of an adhesive sheet (pressure sensitive adhesive sheet).

Next, a contact hole 30 is formed in a laminate between the gas barrier film 20 and the adhesive layer 24. The contact hole 30 is formed in a position corresponding to the electrode 14b of the electronic element 14 to be sealed.

The contact hole 30 may be formed according to a known method. Examples of the known method include punching processing and laser processing. Among these, from the viewpoint of preventing damage to a gas barrier layer of the gas barrier film 20, laser processing is preferably used.

A diameter X of the contact hole 30 may be suitably set according to the size or the like of the electronic element 14. The diameter X of the contact hole 30 will be described later.

The contact hole 30 is basically cylindrical. However, the contact hole 30 is not necessarily cylindrical, and contact holes having various shapes such as an elliptic cylindrical shape, a square tubular shape, and an amorphous tabular shape can be used. The diameter of the contact hole 30 may be changed in the height direction, and examples of such a shape include a truncated cone shape, a truncated pyramid shape, and a shape formed by bonding upper surfaces of two truncated cones to each other. In this case, a cylinder that is inscribed in the contact hole 30 (that is, the smallest diameter) is assumed, and the diameter of this cylinder may be set as the diameter X of the contact hole 30.

It is preferable that the contact hole 30 is formed to have a certain distance between the contact hole 30 and the electronic element main body 14a when the contact hole 30 and the substrate 12 are laminated on each other.

The contact hole 30 is filled with the take-out wire 26. However, since the gas barrier properties of the take-out wire 26 are inferior to those of the gas barrier film 20, there is a possibility that moisture having passed through the take-out wire 26 enters the adhesive layer 24 and reaches the electronic element main body 14a.

On the contrary, when the contact hole 30 is formed to have a certain distance between the contact hole and the electronic element main body 14a, it is possible to prevent moisture, which has passed through the take-out wire 26 and enters the adhesive layer 24, from reaching the electronic element main body 14a.

Here, the time at which the moisture reaches the electronic element main body 14a depends on the temperature and humidity environment and the distance between the contact hole 30 and the electronic element main body 14a.

Accordingly, from the above-described viewpoint, the distance between the contact hole 30 and the electronic element main body 14a may be suitably set such that required durability can be obtained.

In addition to this, as shown in the lower left stage of FIG. 2, the substrate 12 on which one or more electronic elements 14 (electronic element main bodies 14a and electrodes 14b) are formed is prepared. Projections 32 formed of an electroconductive material are formed on the electrodes 14b of the electronic elements 14 by being aligned with the contact holes 30 formed in the laminate of the gas barrier film 20 and the adhesive layer 24. Alternatively, the contact holes 30 may be formed in the laminate of the gas barrier film 20 and the adhesive layer 24 by being aligned with the projections 32.

The projections 32 may be formed using a known method according to the material of forming, the projections 32 or the size thereof. Examples thereof include a method of filling a contact hole with metal paste such as silver paste or gold paste, performing molding as necessary, and drying and optionally further curing the paste, a method of printing using metal paste, and a method of utilizing an ink-jet for which an electroconductive ink is used.

The shape of the projection 32 is not particularly limited and various shapes such as a columnar shape and a conical shape can be used as long as the projection 32 is erected from the electrode 14b.

Preferred examples of the shapes of the projection 32, of which the sizes are gradually decreased (decrease in diameter) toward the upside, include a cone shape, a truncated cone shape, a pyramid shape, a truncated cone shape with a curved upper surface, and a cone shape with a curved upper surface. It is preferable that the projection 32 is formed to have a shape whose size is gradually decreased toward the upside from the viewpoints that the projection 32 is easily formed, the projection 32 is easily inserted into the contact hole 30, and bubbles are unlikely to enter when the gas barrier film 20 is pressure-bonded.

A height Y of the projection 32 may be suitably set according to the size or the like of the electronic element 14. The height Y of the projection 32 will be described later.

After the contact holes 30 are formed in the laminate of the gas barrier film 20 and the adhesive layer 24 and the projections 32 are formed on the electronic elements 14, as shown on the right side of FIG. 2, the contact holes 30 and the projections 32 are aligned with each other, the adhesive layer 24 and the surface on which the electronic elements 14 are formed are allowed to face each other, and the laminate of the gas barrier film 20 and the adhesive layer 24 and the substrate 12 are laminated on each other. Next, as shown in the upper stage of FIG. 3, the gas barrier film 20 and the substrate 12 are pressure-bonded to each other.

Here, according to the method of manufacturing an electronic device, the electronic device has the projections 32, and the diameter X [μm] of the contact hole 30, the height Y [μm] of the projection 32, and the thickness L [μm] of the adhesive layer 24 before pressure-bonding satisfy the following Expression (1) and Expression (2).

0<\sqrt{X}<\sqrt{5000} Expression (1)

Y>−0.018×L×(\sqrt{X}−\sqrt{5000}) Expression (2)

In the method of manufacturing an electronic device of the present invention, with the above-described configuration, it is possible to prevent the contact holes from being filled with the adhesive layer 24 and to form the take-out wire 26 for stably connecting the electrodes 14b and an external device even in a case where the electronic device is small and the contact holes 30 are fine.

As described above, in a case where a small electronic element such as an IC tag is sealed with a gas barrier film, it is considered that a contact hole is formed in the gas barrier film and then a take-out wire for connecting the electronic device to an external device through the contact hole is provided. In a case where an electronic element is small, the contact hole also needs to be small according to the size of the electronic element. When the effective area of the device is considered, it is preferable that the contact hole is small even in a case where the electronic element is large.

Here, as described in JP2011-62958A, adhesion of the gas barrier film on which the adhesive layer is formed to the substrate on which the electronic element is formed is typically performed by pressure-bonding, in which the gas barrier film and the substrate are laminated on each other and pressed against each other. In addition, the adhesive layer is heated or irradiated with light as necessary when the pressure-bonding is performed.

When the gas barrier film and the substrate are pressure-bonded to each other, the adhesive layer (adhesive) moves so as to fill the contact hole. However, the contact hole is blocked due to the pressure-bonding of the gas barrier film and the substrate in a case where the contact hole is small, and thus a wire cannot be taken out.

On the contrary, according to the method of manufacturing an electronic element of the present invention, a projection is formed on the electrode 14b of the electronic element 14, and the diameter X of the contact hole 30 (the size X of the contact hole 30), the height Y of the projection, and the thickness L of the adhesive layer 24 before the pressure-bonding satisfy Expression (1) and Expression (2).

In this manner, as shown in the upper stage of FIG. 3, even when the gas barrier film 20 and the substrate 12 are pressure-bonded to each other and the adhesive layer 24 moves so as to fill the contact holes 30, the projections 32 are present in the contact holes 30 in a state of projecting from the adhesive layer 24. Therefore, as described below, the take-out wires 26 respectively connected to the electrodes 14b can be stably formed by filling the contact holes 30 with an electroconductive material.

According to the examination conducted by the present inventors, the possibility that the contact hole 30 is filled with the adhesive layer 24 decreases as the diameter X of the contact hole 30 increases. Further, the possibility that the contact hole 30 is filled with the adhesive layer 24 decreases as the height Y of a projection increases. In addition, the possibility that the contact hole 30 is filled with e adhesive layer 24 decreases as the thickness L of the adhesive layer 24 decreases.

In a case where Expression (1) is not satisfied, in other words, in a case where the diameter X of the contact hole 30 is 5000 μm or greater, there is a problem in that the contact hole cannot be formed in the small-sized electronic device 10. Further, in the case where the diameter X of the contact hole 30 is 5000 μm or greater, even when the gas barrier film 20 and the substrate 12 are pressure-bonded to each other, the possibility that the contact hole 30 is filled with the adhesive layer 24 is extremely small and thus the formation of the projection 32 becomes meaningless.

Further, in a case where the diameter X of the contact hole 30, the height Y of the projection, and the thickness L of the adhesive layer 24 do not satisfy Expression (2), the height Y of the projection is extremely small with respect to the diameter X of the contact hole 30 and the thickness L of the adhesive layer 24. Accordingly, the projection 32 is filled with the adhesive layer 24 when the gas barrier film 20 and the substrate 12 are pressure-bonded to each other, the take-out wire 26 connected to the electrode 14b cannot be formed.

Basically, the diameter X of the contact hole 30, the height Y of the projection 32, and the thickness L of the adhesive layer 24 are not limited as long as the following Expression (1) and Expression (2) are satisfied.

Further, it is preferable that the height Y of the projection 32 is greater than the thickness L of the adhesive layer 24. When the height Y of the projection 32 is set to be greater than the thickness L of the adhesive layer 24, it is possible to prevent the projection 32 from being buried by the adhesive layer 24 and to stably form the take-out wire 26 connected to the electrode 14b.

It is preferable that the maximum size of the projection 32 is smaller than the diameter X of the contact hole 30 (the size of the contact hole). When the maximum size of the projection 32 is set to be smaller than the diameter X of the contact hole 30, the projection 32 can be suitably inserted into the contact hole 30 and the take-out wire 26 connected to the electrode 14b can be stably formed.

Moreover, the size of the projection 32 indicates the size of the direction perpendicular to the height of the projection 32 as described above. In other words, in a case where the projection 32 has a cone shape or a truncated cone shape, the size of the bottom surface indicates the maximum size of the projection 32.

It is preferable that the thickness L of the adhesive layer 24 is set to be small within the range in which the adhesive force can be sufficiently maintained. When the thickness L of the adhesive layer 24 is set to be small, it is possible to suppress infiltration of moisture from an end portion of the adhesive layer 24 and to reliably prevent the projection 32 from being buried by the adhesive layer 24.

As described above, after the contact holes are formed in the laminate of the gas barrier film 20 and the adhesive layer 24 and the projections are formed on the electronic elements 14, as shown on the right side of FIG. 2, the contact holes 30 and the projections 32 are aligned with each other, and the laminate of the gas barrier film 20 and the adhesive layer 24 and the substrate 12 are laminated on each other. Next, as shown in the upper stage of FIG. 3, the gas barrier film 20 and the substrate 12 are pressure-bonded to each other (the gas barrier film 20 and the substrate 12 are pressed against each other). In addition, the adhesive layer 24 is heated (heated and pressure-bonded) or irradiated with light as necessary when the gas barrier film 20 and the substrate 12 are pressure-bonded.

Subsequently, as shown in the middle stage of FIG. 3, the contact holes 30 are filled with an electroconductive material such that the contact holes 30 are completely buried and then the take-out wires 26 for connecting the electronic element 14 to an external device are formed.

Here, when the gas barrier film 20 and the substrate 12 are pressure-bonded to each other, the adhesive layer 24 moves so as to fill the contact holes 30. However, according to the method of manufacturing an electronic device of the present invention, as described above, the projections 32 are formed, and the diameter X [μm] of the contact hole 30, the height Y [μm] of the projection 32, and the thickness L [μm] of the adhesive layer 24 before the pressure-bonding satisfy Expression (1) and Expression (2).

Accordingly, as shown in the upper stage of FIG. 3, the projections 32 are not buried by the adhesive layer 24 even when the gas barrier film 20 and the substrate 12 are pressure-bonded to each other and the adhesive layer 24 moves so as to fill the contact holes 30. In other words, the projection 32 connected to the electrode 14b is exposed to the inside the contact hole 30. Accordingly, the projection 32 and the electroconductive material are connected to each other by filling the contact hole 30 with the electroconductive material and thus the take-out wire 26 connected to the electrode 14b of the electronic element 14 can be formed.

Further, a size varying portion in the take-out wire 26 formed by filling the contact hole 30 with the electroconductive material is formed by the adhesive layer 24 being moved to the contact hole 30 when the gas barrier film 20 and the substrate 12 are pressure-bonded to each other. For example, in a case where the projection 32 has a shape whose size is gradually decreased toward the upside, such as a cone shape, the take-out wire 26 which has the above-described narrowed portion is formed.

The gas barrier film 20 and the substrate 12 may be pressure-bonded to each other according to a known method. For example, in a case of using the RtoR system, the gas barrier film 20 and the substrate 12 may be continuously pressure-bonded using a pair of pressing rollers.

Further, the pressing force may be suitably set such that the gas barrier film 20 and the substrate 12 can be properly bonded to each other by the adhesive layer 24 according to the material of forming the adhesive layer 24 or the thickness L thereof.

The filling of the contact hole 30 with the electroconductive material, that is, the formation of the take-out wire 26 may be performed using a known method according to the size of the contact hole 30.

Examples thereof include a method of filling a contact hole with metal paste such as silver paste or gold paste, performing molding as necessary, and drying and optionally further curing the paste, a method of printing using metal paste, and a method of utilizing an ink-jet for which an electroconductive ink is used.

After the electronic device 10 is prepared in the above-described manner as shown in the lower stage of FIG. 3, the electronic device 10 is cut to obtain electronic devices 10a. The electronic device 10 may be cut according to a known method.

Such an electronic device 10a is mounted on various devices, such as a display, by the take-out wire 26 being connected to the substrate on which other electronic devices are formed.

The method of manufacturing an electronic device of the present invention may be a so-called batch type method using the sheet-like substrate 12 on which a plurality of the electronic elements 14 are formed and the sheet-like gas barrier film 20.

However, it is preferable to use a so-called RtoR system using the long substrate 12 on which the electronic elements 14 are formed at predetermined intervals in the longitudinal direction and the long gas barrier film 20. As is well known, the RtoR system is a manufacturing method of transporting a long material to be treated from a material roll formed by winding the long material to be treated in a roll shape, performing various treatments while conveying the material to be treated in the longitudinal direction, and winding the treated material in a roll shape again.

According to the method of manufacturing an electronic device of the present invention, the electronic device 10 can be manufactured with higher productivity by performing at least one of the formation of the adhesive layer 24 on the gas barrier film 20, the formation of the contact hole 30, the formation of the projection 32, the lamination of the substrate 12 and the laminate of the gas barrier film 20 and the adhesive layer 24, or the pressure-bonding of the substrate 12 and the gas barrier film 20 to each other and preferably all the processes using the RtoR system.

Further, it is preferable that each of the electronic devices 10a is cut using the RtoR system.

In the method of manufacturing an electronic device according to a first embodiment of the present invention as illustrated in FIGS. 2 and 3, after the projection 32 is formed on the electronic element 14, the substrate 12 and the gas barrier film 20 are laminated and pressure-bonded to each other.

On the contrary, in the method of manufacturing an electronic device according to a second embodiment of the present invention, before the projection 32 is formed on the electronic element 14, the contact hole 30 and the electrode 14b are aligned with each other, the adhesive layer 24 and the surface on which the electronic element 14 is formed are allowed to face each other, and the substrate 12 and the laminate of the gas barrier film 20 in which the contact hole 30 is formed and the adhesive layer 24 are laminated on each other. Next, the projection 32 is formed on the electrode 14b of the electronic element 14 through the contact hole 30 and then the substrate 12 and the gas barrier film 20 are pressure-bonded to each other.

Even with this configuration, when the projection 32 is formed and the diameter X [μm] of the contact hole 30, the height Y [μm] of the projection 32, and the thickness L of the adhesive layer 24 before the pressure-bonding satisfy Expression (1) and Expression (2), the electronic device 10 that includes the take-out wire 26 connected to the electrode 14b, as illustrated in FIG. 1, can be stably manufactured using the same method as the method of manufacturing an electronic device illustrated in FIGS. 2 and 3.

Hereinbefore, the method of manufacturing an electronic device and the electronic device of the present invention have been described in detail, but the present invention is not limited to the above-described examples and various improvements and modifications can be made within the range not departing from the scope of the present invention.

### EXAMPLES

Hereinafter, the present invention will be described in more detail with reference to specific examples.

<Preparation of Gas Barrier Film 20>

A PET film (COSMO SHINE, manufactured by Toyobo Co., Ltd.) having a thickness of 75 μm was prepared as a support. A plasma treatment was applied to this support.

The surface of the support which was subjected to the plasma treatment was coated with a polymerizable composition containing the following polymerizable compound, a polymerization initiator (Esacure KTO46, manufactured by Lamberti Specialty Chemical Co., Ltd.), and 2-butanone such that the dried film thickness was set to 2000 nm, thereby forming a film. This film was irradiated with ultraviolet rays with an irradiation dose of 0.5 J/cm² in a nitrogen atmosphere with an oxygen content of 100 ppm or less, and then a first organic layer was prepared.

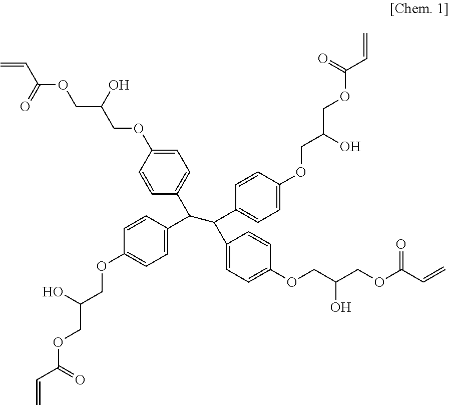 [SMILES: C=CC(=O)OCC(O)COc1ccc(C(c2ccc(OCC(O)COC(=O)C=C)cc2)C(c2ccc(OCC(O)COC(=O)C=C)cc2)c2ccc(OCC(O)COC(=O)C=C)cc2)cc1]

A silicon nitride film (containing oxygen and hydrogen in the film) having a thickness of 40 nm was formed, as an inorganic layer, on the first organic layer using a plasma CVD.

In addition, a second organic layer was formed on the inorganic layer in the same manner as that for the first organic layer and then an organic-inorganic lamination type gas barrier film 20 having a gas barrier layer obtained by alternately laminating an organic layer and an inorganic layer on the support was prepared.

<Formation of Adhesive Layer 24 and Contact Hole 30>

A releasing film was coated with a two-liquid mixed type thermosetting adhesive (EPO-TEK 310, manufactured by Daizo Nichimoly Co., Ltd.) so as to have a desired film thickness and the resulting film was transferred to the gas barrier film 20 prepared in the above-described manner, thereby forming the adhesive layer 24.

Two contact holes 30 were formed in the laminate of the gas barrier film 20 and the adhesive layer 24 formed in the above-described manner. Further, contact holes having diameters of 50 μm and 100 μm were formed by laser processing and the contact hole 30 having a diameter of 200 μm or greater was formed by punching processing using a punch and a die.

### Example 1

Stripe electrodes for a test were formed on the substrate 12 made of a PET film (COSMO SHINE, manufactured by Toyobo Co., Ltd.) having a thickness of 75 μm.

The contact holes 30 were aligned with two stripe electrodes and then substantially conical projections 32, each of which had a bottom surface having a diameter of 50 μm, were formed. The projections 32 were formed with silver paste using a dispenser. Further, the diameter of the bottom surface of each projection 32 was adjusted by changing the nozzle diameter of the dispenser. In addition, the height of each projection 32 was adjusted by changing the coating amount of the silver paste.

The projections 32 and the contact holes 30 were aligned with each other, the substrate 12 and the gas barrier film 20 were laminated and pressure-bonded to each other using a rubber roller, and then the resultant was heated and cured.

After the curing, the contact holes 30 were filled with silver paste, the take-out wires 26 were formed, and conduction between two take-out wires 26 were confirmed.

This conduction test was performed by variously changing the thickness L of the adhesive layer 24, the diameter X of the contact hole 30, and the height Y of the projection 32. Further, as described above, each projection 32 has a substantially conical shape in which the diameter of the bottom surface is 50 μm.

The results are listed in the following tables. When conduction between two take-out wires 26 was confirmed, this case was written as “OK”. In addition, when conduction between two take-out wires 26 was not confirmed, this case was written as “NG”.

TABLE 1
Diameter of bottom surface of projection: 50 μm
Thickness L of adhesive layer: 100 μm

| | | Diameter X of contact hole [μm] | | | | | | | |
|---|---|---|---|---|---|---|---|---|---|
| | | 50 | 100 | 200 | 500 | 1000 | 2000 | 5000 | 10000 |
| Height Y [μm] | No. | NG | NG | NG | NG | NG | NG | NG | OK |
| of projection | projections | | | | | | | | |
| | 10 | NG | NG | NG | NG | NG | NG | OK | OK |
| | 50 | NG | NG | NG | NG | NG | OK | OK | OK |
| | 100 | NG | NG | NG | OK | OK | OK | OK | OK |
| | 200 | OK | OK | OK | OK | OK | OK | OK | OK |

| | | | | | | | | |
|---|---|---|---|---|---|---|---|---|
| Numerical value of | 114.5 | 109.3 | 101.9 | 86.9 | 70.4 | 46.8 | 0 | −52.7 |
| Expression (2) | | | | | | | | |

TABLE 2
Diameter of bottom surface of projection: 50 μm
Thickness L of adhesive layer: 50 μm

| | | Diameter X of contact hole [μm] | | | | | | | |
|---|---|---|---|---|---|---|---|---|---|
| | | 50 | 100 | 200 | 500 | 1000 | 2000 | 5000 | 10000 |
| Height Y [μm] | NG | NG | NG | NG | NG | NG | NG | OK | OK |
| of projection | projections | | | | | | | | |
| | 10 | NG | NG | NG | NG | NG | NG | OK | OK |
| | 50 | NG | NG | NG | OK | OK | OK | OK | OK |
| | 100 | OK | OK | OK | OK | OK | OK | OK | OK |
| | 200 | OK | OK | OK | OK | OK | OK | OK | OK |

| | | | | | | | | |
|---|---|---|---|---|---|---|---|---|
| Numerical value of | 57.2 | 54.6 | 50.9 | 43.5 | 35.2 | 23.4 | 0 | −26.4 |
| Expression (2) | | | | | | | | |

TABLE 3
Diameter of bottom surface of projection: 50 μm
Thickness L of adhesive layer: 10 μm

| | | Diameter X of contact hole [μm] | | | | | | | |
|---|---|---|---|---|---|---|---|---|---|
| | | 50 | 100 | 200 | 500 | 1000 | 2000 | 5000 | 10000 |
| Height Y [μm] | No. | NG | NG | NG | NG | NG | NG | OK | OK |
| of projection | projections | | | | | | | | |
| | 10 | NG | NG | NG | OK | OK | OK | OK | OK |
| | 50 | OK | OK | OK | OK | OK | OK | OK | OK |
| | 100 | OK | OK | OK | OK | OK | OK | OK | OK |
| | 200 | OK | OK | OK | OK | OK | OK | OK | OK |

| | | | | | | | | |
|---|---|---|---|---|---|---|---|---|
| Numerical value of | 11.4 | 10.9 | 10.2 | 8.7 | 7.0 | 4.7 | 0 | 5.3 |
| Expression (2) | | | | | | | | |

As shown in Tables 1 to 3, according to the present invention in which the projections 32 are formed and the diameter X [μm] of the contact hole 30, the height Y [μm] of the projection 32, and the thickness L [μm] of the adhesive layer 24 before the pressure-bonding satisfy Expression (1) and Expression (2), for example, a take-out wire connected to an electrode can be formed even when a contact hole is fine with a diameter of 100 μm or 200 μm.

0<\sqrt{X}<\sqrt{5000} Expression (1)

Y>−0.018×L×(\sqrt{X}−\sqrt{5000}) Expression (2)

Further, when the contact hole 30 has a diameter of 5000 μm or greater, the electronic device becomes unnecessarily large regardless of whether the conduction has been confirmed and the formation of the projection 32 on the electrode 14b of the electronic element 14 becomes meaningless. As the result, the object of the present invention to form a take-out wire connected to the electrode 14b of the electronic element 14 in correspondence with the contact hole 30 with a size which can be filled with the adhesive layer 24 that forms the projection 32 cannot be achieved as described above.

From the above-described results, the effects of the present invention are evident.

The present invention can be suitably applied to electronic devices such as organic EL displays and organic TFTs.

### EXPLANATION OF REFERENCES

10: electronic device

12: substrate

14: electronic element

14a: electronic element main body

14b: electrode

20: gas barrier film

24: adhesive layer

26: take-out wire

30: contact hole

32: projection

What is claimed is:

1. A method of manufacturing an electronic device comprising:
a process of forming an adhesive layer on a gas barrier film and forming a contact hole penetrating through the gas barrier film and the adhesive layer;
a process of forming a projection having electroconductivity on an electrode of an electronic element on a substrate provided with at least one electronic element; and
a process of aligning the contact hole with the projection, allowing the adhesive layer and the surface on which the electronic element is formed to face each other, laminating the substrate and the gas barrier film on each other such that the substrate and the gas barrier film are pressure-bonded to each other,
wherein the following Expressions (1) and (2) are satisfied when the size of the contact hole is set as X [μm], the height of the projection is set as Y [μm], and the thickness of the adhesive is set as L [μm].
0<\sqrt{X}<\sqrt{5000} Expression (1)
Y>−0.018×L×(\sqrt{X}−\sqrt{5000}) Expression (2)

2. The method of producing an electronic device according to claim 1,
wherein the size of a largest portion of the projection is smaller than the size of the contact hole.

3. The method of manufacturing an electronic device according to claim 1,
wherein the height of the projection is greater than the thickness of the adhesive layer.

4. The method of manufacturing an electronic device according to claim 1,
wherein the size of the projection becomes gradually smaller toward the upside in a height direction.

5. The method of manufacturing an electronic device according to claim 1, further comprising:
a process of filling the contact hole with an electroconductive material.

6. The method of manufacturing an electronic device according to claim 1,
wherein the gas barrier film and the substrate have flexibility.

7. The method of manufacturing an electronic device according to claim 1,
wherein at least one of the formation of the adhesive layer, the formation of the contact hole, the formation of the projection, the lamination of the substrate and the gas barrier film on each other, or the pressure-bonding of the substrate and the gas barrier film to each other is performed using a lone substrate and a long gas barrier film while at least one of the substrate or the gas barrier film is conveyed in a longitudinal direction.

8. A method of manufacturing an electronic device comprising:
a process of forming an adhesive layer on a gas barrier film and forming a contact hole penetrating through the gas barrier film and the adhesive layer;
a process of aligning the contact hole with an electrode of an electronic element on a substrate provided with at least one electronic element, allowing the adhesive layer and the surface on which the electronic element is formed to face each other, and laminating the substrate and the gas barrier film on each other;
a process of forming a projection having electroconductivity on the electrode of the electronic element inside the contact hole; and
a process of pressure-bonding the substrate and the gas barrier film to each other,
wherein the following Expressions (1) and (2) are satisfied when the size of the contact hole is set as X [μm], the height of the projection is set as Y [μm], and the thickness of the adhesive is set as L [μm].
0<\sqrt{X}<\sqrt{5000} Expression (1)
Y>−0.018×L×(\sqrt{X}−\sqrt{5000}) Expression (2)

9. The method of producing an electronic device according to claim 8,
wherein the size of a largest portion of the projection is smaller than the size of the contact hole.

10. The method of manufacturing an electronic device according to claim 8,
wherein the height of the projection is greater than the thickness of the adhesive layer.

11. The method of manufacturing an electronic device according to claim 8,
wherein the size of the projection becomes gradually smaller toward the upside in a height direction.

12. The method of manufacturing an electronic device according to claim 8, further comprising:
a process of filling the contact hole with an electroconductive material.

13. The method of manufacturing an electronic device according to claim 8,
wherein the gas barrier film and the substrate have flexibility.

14. The method of manufacturing an electronic device according to claim 8,
wherein at least one of the formation of the adhesive layer, the formation of the contact hole, the formation of the projection, the lamination of the substrate and the gas barrier film on each other, or the pressure-bonding of the substrate and the gas barrier film to each other is performed using a lone substrate and a long gas barrier film while at least one of the substrate or the gas barrier film is conveyed in a longitudinal direction.

15. An electronic device comprising:
a substrate;
at least one electronic element which is formed on the substrate;
a gas barrier film which seals the electronic element;
an adhesive layer which bonds the gas barrier film to the substrate;
a contact hole which penetrates through the gas barrier film and the adhesive layer and is formed in a position corresponding to an electrode of the electronic element; and
a take-out wire which passes through the contact hole and is connected to the electrode of the electronic element,
wherein the contact hole is filled with the take-out wire and the take-out wire includes a size varying portion.

16. The electronic device according to claim 15,
wherein the take-out wire includes a narrowed portion which becomes gradually smaller toward the upside and becomes gradually larger toward the upside from a smallest portion.

17. The electronic device according to claim 15,
wherein a plurality of the electronic elements are formed on the substrate.
